(19) United States
(12) Patent Application Publication
(10) Pub. No.: US 2009/0311346 A1
(43) Pub. Date: Dec. 17, 2009

(54) METHOD FOR TREATING ACNE

(76) Inventor: Neal Schwartz, Ellicott City, MD (US)

Correspondence Address: THE LAW OFFICES OF ANTHONY L. MEOLA, 2500 WESTCHESTER AVENUE, SUITE 401, PURCHASE, NY, 10577, US

(21) Appl. No.: 12/482,088

(22) Filed: Jun. 10, 2009

Related U.S. Application Data
(60) Provisional application No. 61/060,801, filed on Jun. 11, 2008.

(51) Int. Cl. A61K 33/04; A61P 17/10

(52) U.S. Cl. 424/705

(57) ABSTRACT

A novel method for treating acne in an affected area of skin where the method includes having application steps for preparing the affected area of skin, cleansing the affected area of skin and protecting the affected area of skin. The method includes determining a sensitivity level and an irritation level of the affected area of skin to calculate several time periods for applying various cleansers, blends, and lotions to maximize results dependent upon the user's condition.

### CROSS REFERENCE TO RELATED APPLICATIONS

The present application relates to and takes priority from co-pending U.S. Provisional Application Ser. No. 61/060,801, filed on Jun. 11, 2008 and entitled “A COMPOUND AND METHOD FOR TREATING ACNE”, which application is hereby incorporated by reference its entirety.

### BACKGROUND OF THE INVENTION

1. Field of the Present Invention

The present invention relates generally to a compound and method for treating acne.

2. Description of the Related Art

Acne is a disease of the pilosebaceous unit or “pores” (i.e. the structure containing the hair follicle, the hair, and the oil glands. Acne affects males and females at any age but occurs more frequently in the teenage years. The disease may present as a mild nuisance with occasional pimples and plugged pores or severe, with massive inflammation and scarring. The most severe forms of acne occur more frequently in males but the disease tends to persist more frequently in females. Acne usually occurs on the face, chest, arms, and back. It may be short lived or persistent for decades. The psychological effects of Acne range from mild to complete incapacitation.

Increased production of sebum in predisposed individuals is the starting point of acne. (Sebum is an oily substance released by the Sebaceous glands in the skin which helps to maintain hair and skin's moisture and elasticity at normal levels. The sebaceous glands are largest and most numerous on the face, back, chest, and upper outer arms). As a result of the increase in sebum, a normal skin bacterium called Propionibacterium acnes proliferates and begins to alter the sebum's content and the lining of the follicle.

The alteration of the lining of the follicle makes the follicular canal more adherant and also causes the cells of the follicle to increase their production of Keratin. (Keratin is a structural protein that is found in hair, skin, and nails). The mixture of excess keratin and newly changed sebum adhere to the walls of the altered canal and form a plug, known as a microcomedo. All other acne lesions are a consequence of the initial plugging of the follicular canal. Highly inflammatory lesions like pustules, nodules, and cysts result from rupture of the follicular canal with release of its contents into the deeper layers of skin and subsequently causing an intense foreign body reaction.

Acne is an inherited disease. Hormones have been shown to influence oil gland secretion. Other causative factors such as stress and diet have been suspected and debated over for decades but no clear consensus among the literature has been universally accepted. Hormone secreting tumors or measurably overactive glands are less common causes of severe acne. These causes can be ruled out quickly by bloodwork and imaging studies.

Across all genres of medical and healing practices, this chronic condition has been riddled with relapse, fear, and frustration. The failure rate has been masked by years of massive promotion of acne solutions which often deliver unfulfilled promises to those who are suffering the most. These broken promises have created many common misconceptions about acne that this breakthrough practice intends to clear up.

Modern acne treatments have many flaws, including not properly accounting for skin sensitivities and true skin allergies. In addition, as skin sensitivity can change over time, many chronic acne patients have increasingly overly sensitive skin secondary to years of prior art products, oily skin, and active inflammation.

Throughout the last several decades, one acne treatment that has shown acceptable success rates is a powerful oral medication called isotretinoin. Although very powerful, there are numerous short term and possible long term side effects makes it a less desirable option for many health conscious individuals. Other popular oral and topical treatments have only had success in varying degrees and have thus established a wary new trend in the treatment of Acne. A game of trial and error in which patients often become their own unsuccessful practitioners and, months later, find themselves in a deeper spiral of failure. There is also a rising consciousness and anxiety about the types of oral medications we are putting in our bodies. Presently, patients are seeking more natural and holistic ways of healing. In this environment acne has proven to be a very difficult challenge.

There exists a need for a treatment and prevention solution to all cases of acne without the use of oral medications.

### SUMMARY OF THE INVENTION

The present invention includes a method for treating acne.

In a first aspect, the present invention include a method for treating acne including preparing affected skin for treatment by using steam from a pot of boiling water and a towel containing water at a temperature between about 105 degrees Fahrenheit and about 110 degrees Fahrenheit over the affected skin for at least 5 minutes. Some embodiments include exposing the skin to running water a temperature between about 105 degrees Fahrenheit and about 110 degrees Fahrenheit for at least 5 minutes. In some embodiments include submersing the affected skin in water at temperature between about 105 degrees Fahrenheit and about 110 degrees Fahrenheit for at least 5 minutes. In some embodiment an additional step of removing makeup is necessary before the preparation step. Using makeup removing cloths or a non-cleansing makeup remover to remove makeup is an alternative. In some embodiment using the medicated cleansers to remove makeup may implemented dependent upon the irritate level and sensitivity of the affected skin.

The method further includes applying between about 1 cubic centimeter (“cc”) and about 5 cc's of a cleanser to the affected skin, while the affected skin is wet from the preparing step, for a maximum of ten seconds. The method further includes removing the cleanser from the affected skin with water or wet towel.

The method further includes applying between about 1 cubic centimeter (“cc”) and about 5 cc's of a blend to the affected skin, while the affected skin is wet from the removing step, for a maximum of 30 seconds. The method further includes removing the blend from the affected skin with water or wet towel.

The method further includes applying between about 1 cubic centimeter (“cc”) and about 5 cc's of a lotion to the affected skin, while the affected skin is wet from the removing step, for a maximum of 60 seconds. The method further includes removing the lotion from the affected skin with water and dry with a towel.

In some embodiments the method further includes applying about 1 cc of a blend to affected areas for at least 12 hours.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the Figures, wherein:

FIG. 1. shows a flow diagram of a method according to one embodiment the present invention;

FIG. 2 shows an expanded view of method steps according to one embodiment of the present invention;

FIG. 3 shows an expanded view of additional method steps according to one embodiment of the present invention;

FIG. 4 shows an expanded view of yet additional method steps according to one embodiment of the present invention.

FIG. 5 shows an expanded view of still yet additional method steps according to one embodiment of the present invention

### DETAILED DESCRIPTION OF THE PREFERRED EMBODIMENTS

The present disclosure will now be described more fully with reference to the Figures in which an embodiment of the present disclosure is shown. The subject matter of this disclosure may, however, be embodied in many different forms and should not be construed as being limited to the embodiments set forth herein.

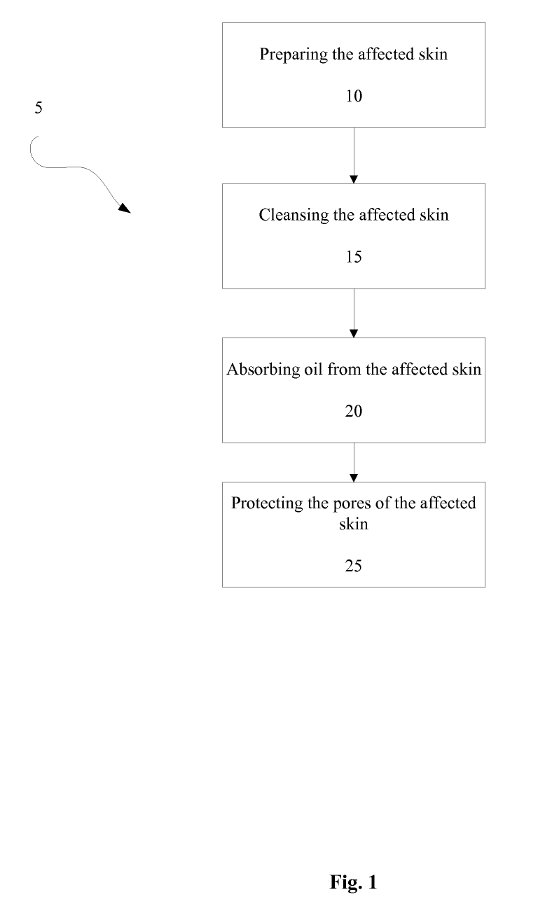

Referring now to the Figures, where in FIG. 1 there is shown the method for treating acne 5 including various method steps according to one embodiment of the present invention. The method includes generally preparing the affected skin 10, cleansing the affected skin 15, absorbing oil from the affected skin 20 and protecting the pores of the affected skin 25.

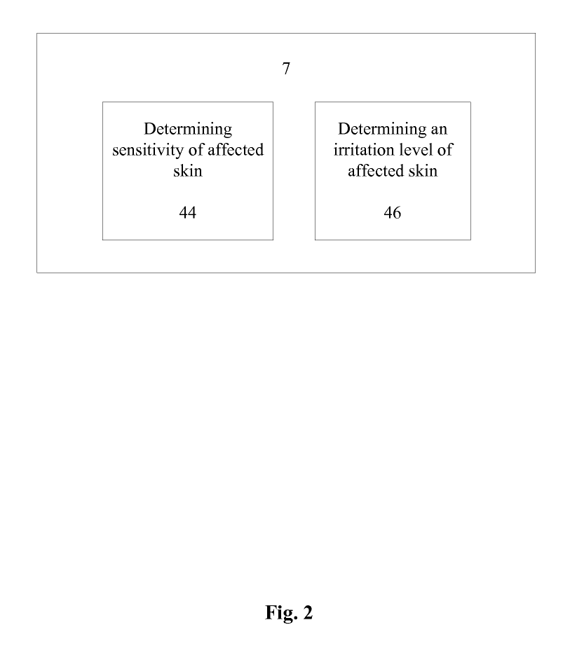

There are many ways to prepare the skin for treatment. The preparing step will be dependent upon the type of skin the user has, the level of sensitivity and the level of irritation of the affected skin before the present invention is implemented. Determining the level of sensitivity 44, determining an irritation level of the affected skin 46 can occur before the preparation step or anywhere during the method on an ongoing basis during an evaluation step 7 as shown in FIG. 2. Some are more effective and some are more convenient for users and some are too harsh while others are too weak.

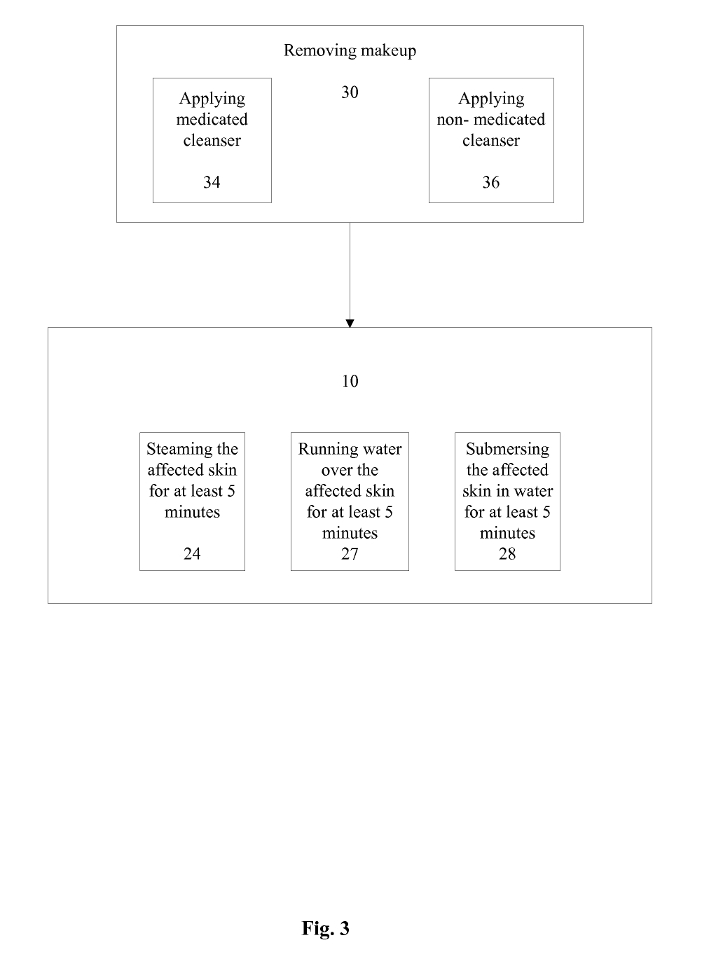

Referring to FIG. 3 there is shown the preparing step 10 with several options. Steaming the affected skin 24 with steam from a pot of water at or above 212 degrees Fahrenheit and preferably a towel covering the affected skin for at least 5 minutes. Additional embodiments include using a facial steamer or a steam room to accomplish the preparing step 10. Some users may run water over the affected area 27. In this embodiment it is preferable that the water is between about 105 degrees Fahrenheit and about 110 degrees Fahrenheit. Finally some user may submerse the affected skin in water, preferably between about 105 degrees Fahrenheit and about 110 degrees Fahrenheit, for at least 5 minutes.

In some embodiments the preparation step may utilize different water temperatures and different exposure times if the user's affected skin is dry, tight, red, irritated, becoming darkened, or wrinkled.

Referring now to FIG. 3 there is shown an alternative embodiment for removing makeup before the preparation step 10. In step 30 the invention includes removing makeup from the affected skin with a medicated cleanser 34. Alternatively the invention may include removing makeup from the affected skin with a non-medicated cleanser 34. The determination to use a medicated or non-medicated cleanser in the makeup removal step 30 is dependent upon the determinations made in step 7. Additionally, the invention may include removing the makeup with a cloth having non-cleansing makeup remover solution Using the medicated cleansers to remove makeup may over-irritate sensitive skin. Less sensitive skin will be able to tolerate the medicated cleansers to remove makeup.

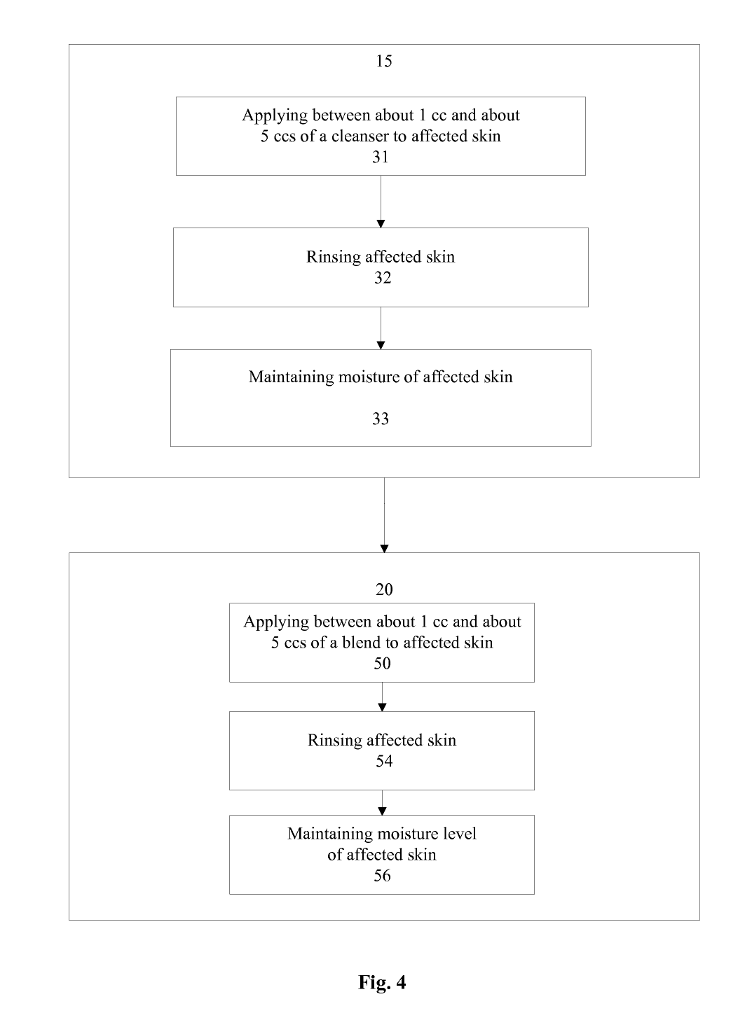

Referring now to FIG. 4 there is shown additional steps in connection with the cleansing step 15 and the absorbing step 20. Preferably, the invention method includes applying between about 1 cubic centimeter (“cc”) and about 5 cc's of a cleanser to the affected skin in step 31. Preferably the applications step should be for no more than 10 seconds before the rinsing step 32 occurs. It is preferably to avoid affected skin areas that get dry or irritated easily, again dependent upon the determinations made in step 7. Ideally the application of the cleanser should be a “passing step” meaning that the user should not try to get completely clean from this step. Hence the application of the cleanser is for between 1 and about 10 seconds and then rinsed off. If the affected skin has a high tolerance, the method may include repeat of the application step 31 to increase amount and time of application.

In all scenarios it is preferable to maintain moisture on the affected skin 33 after the rinsing step, hence the invention provides for leaving the affected skin wet after the rinsing step 32

Importantly, the preferred embodiment provides that the user will avoid applying cleanser on affected skin that is already irritated, red, raw, cracking, overly-wrinkled, itchy, or turning into dark patches. It is critical to master the timing of the application of the various cleaners, blends, and lotions in connection with the determination step 7. The sensitivity and irritation level of the affected skin will directly influence the viability and effectiveness of the present invention. As such the timing of the steps and moisture content of the affected skin at the various steps is critical to the intention.

Step 20 includes absorbing oil from the affected skin. In some embodiment, step 20 also includes applying between about 1 cc and about 5 cc's of a blend to the affected skin 50. Preferably the applying step occurs for between about 1 second and 30 seconds, after which a rinsing step 54 occurs. Again, it is preferable to maintain moisture on the affected skin 56 after the rinsing step 54.

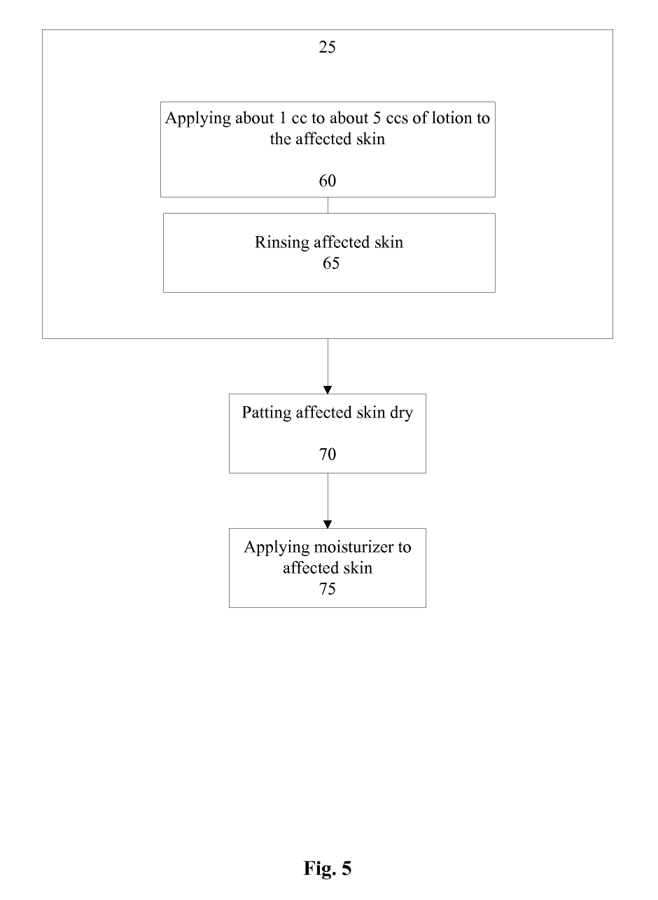

Referring now to FIG. 5, there is shown the protecting the affected skin step 25. Step 25 may include applying between about 1 cc and about 5 cc's of a lotion to the affected skin 60. Preferably the step 60 occurs for between about 1 second and 60 seconds, after which a rinsing step 65 occurs. Preferably, after step 65, the invention includes patting dry the affecting skin 70.

In some embodiments, there is an applying a moisturizer step 75 to the affected skin. This will be dependent upon the sensitivity and irritation level as determined in step 7.

In some embodiments the invention includes an oil absorbing step that lasts overnight or for at least 10 hours. In this embodiment an application of the blend onto the affected skin may occur, preferably avoiding flat dark marks and non-affected skin areas.

In connection with the cleansing step 15 step embodiments the present invention include various cleanser formulations. For example, a gentle cleanser, typically for sensitive skin, may include deionized water, TES lauryl, sulfate, cetyl alcohol, stearyl alcohol, propylene, methylparaben, propylparaben, diazolidinyl, urea and a coloring agent. Another embodiment may include a cleanser with benzoyl-peroxide, great burdock, erysium, citric acid, glycolic acid and zinc, where the benzoyl peroxide is about 2.5%.

In some embodiments the absorbing step 20 includes a blend having sulphur, great burdock, erysium, citric acid, glycolic acid and zinc where the sulphur is about 10%. In some embodiments the protecting step 25 includes a lotion having sulphur, benzoyl-peroxide, great burdock, erysium, citric acid, glycolic acid and zinc, where the sulphur is about 3% and the benzoyl-peroxide 5%.

It will be apparent to one of skill in the art that described herein is a novel method for treating acne. While the invention has been described with reference to specific preferred embodiments, it is not limited to these embodiments. The invention may be modified or varied in many ways and such modifications and variations as would be obvious to one of skill in the art are within the scope and spirit of the invention and are included within the scope of the following claims.

1. A method for treating acne, the method comprising the steps of:
preparing an affected area skin, including exposing the affected area of skin to water for at least 5 minutes;
applying between about 1 cc to about 5 cc's of a cleanser to the affected area of skin for no more than 10 seconds;
applying between about 1 cc to about 5 cc's of a blend to the affected area of skin no more than 30 seconds;
applying between about 1 cc to about 5 cc's of a lotion to the affected area of skin no more than 60 seconds;
rinsing affected area of skin and
patting dry the affected area of skin.

2. The method according to claim 1 where the preparing step further includes steaming the skin with the steam and covering the affected area of skin with a towel for at least 5 minutes.

3. The method according to claim 1 where the preparing step further includes running water between about 105 degrees Fahrenheit and about 110 degrees Fahrenheit over the affected area of least 5 minutes.

4. The method according to claim 1 where the preparing step further includes submersing the affected area of skin in water between about 105 degrees Fahrenheit and about 110 degrees Fahrenheit for at least 5 minutes.

5. The method according to claim 1 further comprising the step of applying a cleanser having between about 2.5% and about 10% benzoyl-peroxide.

6. The method according to claim 5 further comprising the step of applying a cleanser having about 5% sulphur.

7. The method according to claim 1 further comprising the step of applying a blend having about 10% sulphur.

8. The method according to claim 1 further comprising the step of applying a lotion having about the 3% sulphur and the 5% benzoyl-peroxide.

9. The method according to claim 1 further comprising the step of applying an exfoliate having pumice and between about 2.5% and about 10% benzoyl-peroxide.
